(19) United States
(12) Patent Application Publication
(10) Pub. No.: US 2006/0228403 A1
(43) Pub. Date: Oct. 12, 2006

(54) Nutritional supplements for cardiovascular health

(76) Inventor: Karl Arthur Zimmerman, West Chester, PA (US)

Correspondence Address: KARL ARTHUR ZIMMERMAN, 953 CLOOD LANE, WEST CHESTER, PA, 19382, US

(21) Appl. No.: 11/104,123

(22) Filed: Apr. 12, 2005

(51) Int. Cl. A61K 31/714; A61K 31/4415; A61K 31/455; A61K 31/525; A61K 31/51; A61K 31/375; A61K 31/195; A61K 31/202; A61K 31/12; A61K 31/355; A61K 9/127

(52) U.S. Cl. 424/450; 514/52; 514/251; 514/276; 514/350; 514/355; 514/458; 514/560; 514/474; 514/685; 514/562; 514/690

(57) ABSTRACT

An admixture containing as active ingredients omega-3 polyunsaturated fatty acids selected from eicosapentaenoic acid (EPA), docosahexaenoic acid (DHA) and linoleic acid (LNA), curcumin, B complex vitamins selected from folic acid, B6 and B12, antioxidant vitamins selected as vitamin C and vitamin E and ubiquinone. This compound is being designed for the support of cardiovascular health.

### BACKGROUND

Atherosclerotic Coronary Vascular Disease (ASCVD) is currently the largest cause of death in developed countries. This is manifest largely through the ravages of ischemic heart disease and stroke. Many advances in pharmaceutical development, thrombolytic therapy, and interventional cardiology have improved morbidity and mortality in patients who suffer with ASCVD. However, despite these advances; ASCVD remains the number one cause of death in developed countries. This would suggest that despite advances in pharmaceuticals and interventional cardiology that there are factors involved in the development of ASCVD that are currently not being addressed.

The present invention relates to a nutraceutical/nutritional composition for supporting vascular health. The purpose of said invention is to address nutritional deficiencies and insufficiencies in persons with ASCVD. The composition contains at least one ingredient from each of the following families of compounds known to promote vascular health. Specifically at least one ingredient from each of the following families; (a) essential fatty acids, (b) anti-inflammatory agents or inhibitors of arachadonic acid formation (c) agents that promote lowering of plasma homocysteine (d) antioxidant vitamins, (e) free radical scavengers. Simultaneous delivery of these compounds allows for synergistic effects and overall promotion of vascular health. Benefit is also realized by increased compliance with ease of administration.

(a) Omega-3 Fatty Acids

Diets high in saturated fats are associated with elevated cholesterol levels and increased risk of cardiovascular disease and ASCVD. Natives of fishing populations have lower cholesterols and decreased risk of cardiovascular disease. This is thought to be secondary to their high intake of a group of essential fatty acids from fish. These are known as Omega-3 fatty acids or n-3 fatty acids. Specifically eicosapentaenoic acid (EPA) and docosahexaenoic acid (DHA) have been shown to improve endothelial function primarily through elevation of nitric oxide.⁽¹⁾ Omega-3 supplementation has been shown to lower triglyceride levels and very-low lipoprotein (VDL) levels.⁽²⁾ Omega-3 fatty acids have been shown in numerous clinical trials to promote improved cholesterol profiles and recently to improve mortality in patients post myocardial infarction.^{(3,4,5)}

(b) Anti-inflammatory Agents/Inhibitors of Arachadonic Acid Formation

Recent data suggests that chronic inflammation may play a role in the development of atherosclerosis. Studies are ongoing looking at the effect of non-steroidal anti-inflammatory agents and inhibitors of arachadonic acid in the pathogenesis of coronary artery disease. Curcumin's effect on arachadonic acid formation has been well documented. We believe that curcumin may benefit vascular health by decreasing endovascular inflammation and thus promoting vascular health.

(c) Agents that Promote Lowering of Plasma Homocysteine

Folic acid functions as a cofactor in DNA synthesis. Folate donates a methyl group to oxidation at various levels of purine synthesis. During purine synthesis the amino acid methionine is produced from homocysteine. Folate deficiency/insufficiency leads to impaired DNA synthesis and decreased production of methionine. This causes an excess of homocysteine. Elevation of homocysteine has been linked to endothelial damage and increased thrombogenesis.⁽⁶⁾ Elevation of plasma Homocysteine has been clearly shown to be an independent risk factor for the development of premature cardiovascular, cerebrovascular and peripheral vascular disease. Clear risk reductions in mortality and morbidity have been shown by the addition of folic acid to the diet. This coincides with a linear relationship between plasma homocysteine and mortality from ASCVD.⁽⁷⁾

(d) Antioxidant Vitamins

Antioxidant Vitamins are agents that sacrifice themselves to oxygen. Oxidation is the process of cell aging. During this process, chemical compounds known as free radicals are generated. Free radicals contribute to apoptosis and cell death. Antioxidant vitamins prevent free radical production. Ascorbic acid (vitamin C) and alpha-tocopherol (vitamin E) are two widely used antioxidant vitamins. Vitamin C role in providing cardiovascular protection has been shown is several large clinical trials.

(e) Free Radical Scavengers

Certain families of compounds have an important role in scavenging of free radicals. Most notably are members of the ubiquinone family of compounds. Ubiquinone (CoQ10) lowers intracellular free radical concentration by effectively acting as a sink for intracellular free radicals.

### SUMMARY OF THE INVENTION

Optimal benefits for human health are achieved by delivering the admixture as a combination formulation simultaneously in various delivery systems i.e. solid, semisolid, liquid, semi liquid, powder, granular or liposomic form and occurring in tablets, capsules, softgels, powders or liquid, semi liquid form for oral administration. Said compound, being delivered in a fashion that would have an optimal combination of anti-inflammatory -cardiovascular support compounds resulting in an overall support to vascular function.

More specifically the invention relates to a composition comprising (a) at least one omega-3 polyunsaturated fatty acid selected from the group of eicosapentaenoic acid, docosahexaenoic acid and linoleic acid in amounts effective for lowering total cholesterol and supporting cardiovascular health and (b) at least one isomer of the curcuminoid family of compounds (c) at least one B vitamin selected from the group of folic acid, pyridoxine and cyanocobalamin (d) at least one member of the family of water soluble vitamins, known for their antioxidant properties (e) The resulting composition being effective in supporting nutritional insufficiencies in persons with cardiovascular disease or persons interested in maintaining cardiovascular health.

### REFERENCES

1. Overvad V, et al. Coenzyme Q10 in health and disease. Eur J Clin Nutr. 1997;53:764-770.
2. Adler A J, et al. Effect of garlic and fish oil supplementation on serum lipid and lipoprotein concentrations in hypercholesterolemic men. Am J Clin Nutr. 1997;65:445-450.
3. Hu, F. B., et al. Fish and Omega-3 fatty acid intake and risk of coronary heart disease in women. JAMA 2002 Apr. 10; 287:1815-21.
4. Albert, C. M., et al. Blood levels of long chain fatty acids and the risk of sudden death. NEJM 2002 Apr. 11; 346:1113-8.
5. Marchioli, R., et al. Early protection against sudden death by n-3 polyunsaturated fatty acids after myocardial infarction. Circulation. 2002; 105:1897.
6. Puddu P, Homocysteine and the risk of atherothrombotic events. Cardiologia. 1999;44:341-345.
7. Boushey, et al. A quantitative assessment of plasma homocysteine as a risk factor for vascular disease. Probable benefits of increasing folic acid intakes. JAMA. 1995;274.

### REFERENCE READING

### REFERENCE READING Monocytes in the Coronary Circulation of Reperfused Human Myocardium. No Effect of Preoperative Treatment with N-3 Fatty Acids.” Scand J Thorac Cardiovasc Surg 27(2): 81-86.

In a double-blind study 18 patients were randomized to receive a daily dietary supplement of concentrated ethyl ester compound of n-3 fatty acids or placebo (corn oil) for at least 6 weeks before coronary bypass surgery. Three-fold increase of serum eicosapentaenoic acid and 20% reduction of triglyceride levels were found preoperatively in the n-3 group, while the two groups were similar as regards monocyte and platelet counts, mean platelet volume and monocyte activation as expressed by thromboplastin activities. For determination of transcardiac gradients, coronary sinus and aortic blood were sampled preoperatively 5, 10 and 30 minutes after release of the aortic cross-clamp. In both patient groups the monocyte count was lower in coronary sinus than in aortic blood at 5 and 10 minutes, but the differences were not significant. The platelet counts showed no significant change. In vitro stimulation of monocytes, however, evoked significantly (p<0.05) less thromboplastin activity in coronary sinus blood than in aortic blood at all three sampling times, without significant intergroup difference. The monocytes most sensitive to activation presumably were trapped in the reperfused myocardium, and this sequestration was not hindered by pretreatment with n-3 fatty acids.

Baronzio, G. F. Galante, etc. (1998). “Tumor Microcirculation and Its Significance in Therapy: Possible Role of Omega-3 Fatty Acids as Rheological Modifiers.” Med Hypotheses 50(2): 175-182.

Despite the great efforts to find new drugs or devices to suppress cancer cells, attempts to modify microcirculation and therefore the state of tumor cells and their surrounding normal tissues have not been given the attention they deserve. Solid tumors are composed of highly heterogeneous populations of malignant, stromal and inflammatory cells in a continuously adapting extracellular matrix. All of the above components interact and regulate each other to produce distinct microenvironments within the tumor mass. Abnormal microcirculation plays a particular role in the maintenance of this anomalous condition and favors the formation of metastasis, but on the other hand provides the therapist with an important site for intervention. In this brief overview we attempt to outline three aspects: (a) how the anomalous tumor blood flow provokes the nonuniform distribution of oxygen and nutrients within the tumor mass, thus determining different responses to the various cancer therapies; (b) how hemorheology is the clinical parameter most easily modified and (c) how omega-3 essential fatty acids are natural drugs that could be used in this sense beyond their antitumoral properties.

Bruckner, G. (1997). “Microcirculation, Vitamin E and Omega 3 Fatty Acids: An Overview.” Adv Exp Med Biol 415: 195-208.

We have observed significant increases in LDF and similar trends for CBV after FO supplementation in younger subjects (both normolipidemic and hyperlipidemic). In elderly subjects, this trend appears to be reversed unless subjects are supplemented with higher doses of vitamins. E (100 IU/10 KG/day). Our mouse data suggest that dietary vit. E at 100 IU/Kg does not adequately protect against lipid oxidation in vivo or in vitro following an oxidative insult when mice are fed an 8% FO & 2% linoleic acid containing diet. It has been reported that FO significantly lowers triglycerides and VLDL-cholesterol (especially where subjects have higher initial triglyceride values) and tends to increase LDL-cholesterol and Apo-B100. These findings are all the more important because the oxidation of LDL from FO-supplemented subjects caused a time-dependent increase in the ability to facilitate albumin transfer which was not diminished following a 2 month washout (WO). Addition of vitamins. E to the FO supplement prevented this change. These data suggest that FO supplementation without sufficient vitamins. E may be deleterious to the vascular endothelium. The western fat blend supplement appeared to be protective with increased length of supplementation most likely due to increased MONO fatty acids which are resistant to oxidation; vit. E supplementation appeared to have little additional effect. Our combined studies, and those reported by others, suggest that in humans, increased peripheral microcirculatory flow is most likely due to changes in precapillary vascular tone i.e., vasodilation. It is also possible that subtle changes in each of the three variables i.e. blood pressure, blood viscosity and vascular tone when combined may contribute to the significant changes which we have noted as increased LDF or CBV after intervention with dietary n-3 fatty acids. We hypothesize that interactions between dietary fatty acids and vitamins. E alters the ratios of vasoconstrictive-platelet aggregatory/vasodilatory-antiplatelet aggregatory agents (TXA2 and endothelin/PGI2 and nitric oxide), the expression of adhesion molecules (P-selectin and E-selectin) and thereby directly influences the modulation of free radical mediated events between blood elements and the vascular endothelium. Fatty acids of the n3 series may alter these events by favoring the production of vasodilatory compounds and decreased expression of P and/or E-selectins, provided that these highly oxidizable lipids are protected by adequate antioxidants.

Enikeeva, N. A. P. A. Manasova, et. l. (1998). “[Effect of Diet Enriched with Marine Omega-3 Polyunsaturated Fatty Acids on Microcirculation System in Patients with Cardiorespiratory Pathology].” Vopr Pitan(4): 39-41.

387 patients with cardiorespiratory pathology being under the influence of antiatherosclerotic diet were observed to have the positive dynamic of clinical picture of diseases, indices of microcirculatory channel and lipid spectrum of blood. Diet enrichment with polyunsaturated fatty acids omega-3 of sea origin favors the strengthening of corrected influence on lipid metabolism and microcirculatory state improving the miocardial metabolism.

Heizer, M. L., J. S. McKinney, et al. (1992). “The Effect of Dietary N-3 Fatty Acids on in Vivo Platelet Aggregation in the Cerebral Microcirculation.” Thromb Res 68(4-5): 383-391.

Diets enriched in n-3 fish oil have been suggested to decrease coronary artery disease in part through their ability to decrease cyclooxygenase-dependent platelet aggregation. However little is known concerning the effect of n-3 fatty acids on in vivo platelet aggregation. The purpose of these experiments was to determine whether dietary n-3 fatty acids affect the rate at which platelet aggregation occurs in cerebral arterioles. Fish oil (200 mg eicosapentaenoic acid+143 mg docosahexaenoic acid/kg), corn oil or water was given daily by gavage to mice (n=30) for six weeks and then in vivo platelet aggregation was induced by the light plus dye method, which injuries the endothelium. Two additional groups of mice were acutely treated with saline or indomethacin (0.5 mg/kg, ip), with the latter serving as a positive control for therapeutic inhibition of platelet aggregation. Serum thromboxane B2 was analyzed by RIA. All fed groups gained weight equally. Serum thromboxane B2 was decreased by 40% in the fish oil group (p=0.05 vs. corn oil, p=0.07 vs. water). The mean (+/−SE) time to first aggregate in pail arterioles was 101+/−6, 91+/−6 and 101+/−9 seconds in the fish corn oil and water groups, respectively. Indomethacin significantly increased the time to first arteriolar aggregate by 35% (p<0.002) and caused an 80% reduction in serum thromboxane. These studies show dietary fish oil produces a moderate reduction in serum TxB2 level and does not affect arteriolar platelet aggregation whereas indomethacin produces a drastic TxB2 reduction and significantly slows platelet aggregation.(ABSTRACT TRUNCATED AT 250 WORDS)

Karlsson, J., R. Ronneberg, et al. (1997). “Vitamins Q and E, Extracorporal Circulation and Hemolysis.” Mol Cell Biochem 173(1-2): 33-41.

Whole blood vitamin Q (ubiquinone), plasma vitamins Q and E (alpha-(alpha-)tocopherol) and free cholesterol (FC) were studied before (control or base-line value, sample I) and during open chest surgery and extracorporal circulation (samples II-IV) in 10 male IHD patients. Identity existed between control whole blood and plasma ubiquinone. During surgery an increased discrepancy with lower plasma vitamin Q levels were seen. Control plasma vitamins Q, E and FC averaged 0.88+/−0.16 (SE), 12.1+/−2.2 mg×I(−1) and 0.75+/−0.15 g×I(−1). Corresponding molar values were 1.02+/−0.17, 28.1+/−5.1 micromol×I(−1) and 1.94+/−0.74 micromol×I(−1). Vitamin Q and E decreased continuously and averaged 0.64 mg×I(−1) in sample IV (0.74 micromol×I(−1), p<0.001) and 9.4 mg×I(−1) in sample III (21.8 micromol×I(−1), p<0.001). Hemolysis in all sample IV vials, ruined all vitamin E determinations. When normalized for FC (NQ and NE), decreases were found to be 17 (IV) and 12% (III), respectively. Large interindividual variations existed. High control NQ and NE values allowed a larger antioxidant vitamin depletion. High NQ seemed also to be a prerequisite for NE depletion. In addition, signs indicated an active liver vitamin Q release for patients rich in control antioxidant values. It was suggested that the antioxidant vitamin depletion did not prevent from radical trauma to membrane structural lipids (especially omega-3 fatty acids or vitamin F1), less membrane fluidity, erythrocyte fragility and hemolysis.

Lombard, J. H., M. P. Kunert, et al. (1999). “Cytochrome P450 Omega-Hydroxylase Senses O2 in Hamster Muscle, but Not Cheek Pouch Epithelium, Microcirculation.” Am J Physiol 276(2 Pt 2): H503-508.

The goal of this study was to investigate the role of cytochrome P450 omega-hydroxylase in mediating O2-induced constriction of arterioles in the microcirculation of the hamster. Male Golden hamsters were anesthetized with pentobarbital sodium, and the cremaster muscle or cheek pouch was prepared for observation by intravital microscopy. Arteriolar diameters were measured during elevations of superfusate PO2 from approximately 5 to 150 mmHg.

Arteriolar responses to elevated PO2 were determined in the cremaster muscle, in the retractor muscle where it inserts on the cheek pouch, and in the epithelial portion of the cheek pouch. Elevation of superfusion solution PO2 caused a vigorous constriction of arterioles in the cremaster and retractor muscles and in the epithelial portion of the cheek pouch. Superfusion with 10 micromolar 17-octadecynoic acid, a suicide substrate inhibitor of cytochrome P450 omega-hydroxylase, and intravenous infusion of N-methylsulfonyl-12,12-dibromododec-11-enamide, a mechanistically different and highly selective inhibitor of cytochrome P450 omega-hydroxylase, caused a significant reduction in the magnitude of O2-induced constriction of arterioles in the cremaster and retractor muscles. However, arteriolar constriction in response to elevated PO2 was unaffected by 17-octadecynoic acid or N-methylsulfonyl-12,12-dibromododec-11-enamide in the epithelial portion of the cheek pouch. These data confirm that there are regional differences in the mechanism of action of O2 on the microcirculation and indicate that cytochrome P450 omega-hydroxylase senses O2 in the microcirculation of hamster skeletal muscle, but not in the cheek pouch epithelium.

Lyzogub, V. G., T. V. Zaval's'ka, et al. (2001). “[Role of the Skin in Destabilization of Coronary Circulation].” Lik Sprava(5-6): 52-55.

Our objective in this paper was to study diagnostic specificities of the skin secretion lipids fatty acid composition (FAC) in the precardiac region of patients with ischemic heart disease (IHD). Overall ninety two IHD patients who ranged from 34 to 78 years were examined together with fifteen clinically healthy persons. Used in the study were the gas-liquid chromatography techniques. The secured results suggested to us changes in the precardiac region skin lipids FAC in IHD. During the stage of compensation of coronary circulation the secretion of palmitic and docozohexaenic acids was found to have gotten diminished while that of saturated acids was augmented. This suggests to us a positive role of the skin, its negative role being in an increased secretion of monounsaturated fatty acids (MUFA), decreased secretion of representatives of omega-6 polyunsaturated fatty acids (PUFA). During the stage of decompensation the skin has predominantly a negative part (increased secretion of MUFA, docozohexaenic acids, decreased secretion of omega-6PUFA).

Mori, T. A., G. F. Watts, et al. (2000). “Differential Effects of Eicosapentaenoic Acid and Docosahexaenoic Acid on Vascular Reactivity of the Forearm Microcirculation in Hyperlipidemic, Overweight Men.” Circulation 102(11): 1264-1269.

### BACKGROUND

Recent evidence supports differential effects of eicosapentaenoic acid (EPA) and docosahexaenoic acid (DHA), the 2 major omega3 fatty acids of marine origin, on blood pressure in humans and vascular reactivity in adult spontaneously hypertensive rats. We investigated possible differences in the effects of purified EPA or DHA on forearm vascular reactivity in overweight hyperlipidemic men that might contribute to the blood pressure-lowering effects of fish oils.

### METHODS AND RESULTS

With a double-blind, placebo-controlled trial of parallel design, 59 overweight, mildly hyperlipidemic men were randomized to receive 4 g/d purified EPA, DHA, or olive oil (placebo) capsules while continuing their usual diets for 6 weeks. Forearm blood flow (FBF) was measured with venous occlusion, strain-gauge plethysmography during the sequential intra-arterial administration of acetylcholine (7.5, 15, and 30 microg/min), sodium nitroprusside (1.5, 3, and 10 microg/min), norepinephrine (10, 20, and 40 ng/min), a single-dose infusion of N:(G)-monomethyl-L-arginine (L-NMMA) (1 mg/min), and coinfusion of acetylcholine (7.5, 15, and 30 microg/min) and L-NMMA. Forty of the 56 subjects who completed the study underwent FBF measurements. Plasma phospholipid EPA levels increased significantly (P:<0.0001) after supplementation with EPA, and DHA composition increased with DHA supplementation (P:<0.0001). Relative to placebo, DHA, but not EPA, supplementation significantly improved FBF in response to acetylcholine infusion (P:=0.040) and coinfusion of acetylcholine with L-NMMA (P:=0.040). Infusion of L-NMMA alone showed no group differences. DHA significantly enhanced dilatory responses to sodium nitroprusside (P:<0.0001) and attenuated constrictor responses to norepinephrine (P:=0.017).

### CONCLUSIONS

Relative to placebo, DHA, but not EPA, enhances vasodilator mechanisms and attenuates constrictor responses in the forearm microcirculation. Improvements in endothelium-independent mechanisms appear to be predominant and may contribute to the selective blood pressure-lowering effect observed with DHA compared with EPA in humans.

What is claimed is:

1. A composition for use in humans containing either an admixture of/or individual compounds delivered simultaneously in individual dosages of the following compounds delivered as a single mixture formula:
(a) At least one omega-3 polyunsaturated fatty acid or an ester thereof, in an amount effective for enhancing vascular health and promoting a healthy cholesterol profile.
(b) Curcumin in an amount specific for reducing endothelial inflammation and decreasing platelet aggregation.
(c) At least one B vitamin in an amount effective for lowering plasma homocysteine, wherein the B vitamin is selected from a group consisting of folic acid, B6 and B12.

2. The composition in claim 1, wherein at least 50 mg of ascorbic acid (vitamin C) is present.

3. The composition in claim 2, wherein at least 50 I.U. of alpha-tocopherol (vitamin E) is present.

4. The composition in claim 3, wherein at least 10 mg of ubiquinone (CoQ10) is present.
The composition in claim 1 occurring in solid, semisolid, liquid, semi-liquid, powder, granular or liposomic form and occurring in tablets, capsules, gelatin capsules, powders, or suspension form for oral administration.

5. The composition in claim 1 to be administered for nutritional supplementation in persons with cardiovascular disease including hypertension, stroke, transient ischemia, intermittent claudication, coronary artery disease, carotid artery disease, cerebrovascular disease, peripheral artery disease, renal artery disease, diabetes mellitus; (both youth and maturity onset), hyperlipidemia, and homocysteinuria

6. The composition in claim 1 to be administered for nutritional supplementation in persons without any of the disease states claimed in (6) above, but who are concerned about maintaining vascular health.
